(19) United States
(12) Patent Application Publication
(10) Pub. No.: US 2013/0260410 A1
(43) Pub. Date: Oct. 3, 2013

(54) MICROPLATE SAMPLING ADAPTER

(71) Applicants: Annette Helle Johansen, Broenshoej (DK); Jakob Rothe, Skovlunde (DK); Noriko Tsutsumi, Chiba (JP); Tomoko Matsui, Chiba (JP)

(72) Inventors: Annette Helle Johansen, Broenshoej (DK); Jakob Rothe, Skovlunde (DK); Noriko Tsutsumi, Chiba (JP); Tomoko Matsui, Chiba (JP)

(73) Assignee: NOVOZYMES A/S, Bagsvaerd (DK)

(21) Appl. No.: 13/992,541

(22) PCT Filed: Dec. 8, 2011
(86) PCT No.: PCT/EP11/72156

§ 371 Date: Jun. 7, 2013

(30) Foreign Application Priority Data
Dec. 8, 2010 (EP) 10194200.1

(51) Int. Cl. C12Q 1/24

(52) U.S. Cl. CPC C12Q 1/24
USPC 435/30; 435/309.1

(57) ABSTRACT

The present invention relates to adapters for microplates. The adapters allow sampling from a liquid growth medium in a microplate well after a microorganism has formed a dense layer of biomass on the surface of the growth medium. The adapters also allow easy transfer of a surface-grown microorganism from all wells of a microplate in one go, when inserted before the microorganism form the layer and then removed after incubation and layer-formation, which also enables easy sampling of the growth medium in the

### FIELD OF THE INVENTION

The present invention relates to adapters for microplates that allow sampling from a liquid growth medium in the microplate wells even when a microorganism has formed a dense layer of biomass on the surface of the growth medium. The adapter, when fitted to a microplate, presses down the biomass surface-layer in each well so that the liquid medium flows around and above the biomass layer to allow the pipetting of samples from the medium in the wells without having to penetrate the biomass layer.

The adapter also allows easy transfer of biomass from surface-growing microorganisms from all wells in a microplate in one go, when the adapter is fitted on the microplate before the plate is incubated and the microorganism forms a layer of biomass on the surface of the liquid growth medium inside the hollow fingers of the adapter. After incubation, the adapter is lifted from the microplate and the liquid growth medium drains from its protruding fingers into the wells of the microplate leaving only the biomass inside the fingers and the spent supernatant in the wells of the microplate. This allows easy transfer of the biomass, e.g., into another microplate or into deep freeze storage. After incubation and removal of the adapter and the biomass surface layer, the spent supernatant in each microplate well is accessible for easy sampling.

### BACKGROUND OF THE INVENTION

A microtiter plate or microplate is a flat plate with multiple “wells” used as small test tubes. The microplate has become a standard tool in analytical research and clinical diagnostic testing laboratories. A very common usage is in the enzyme-linked immunosorbent assay (ELISA), the basis of most modern medical diagnostic testing.

A microplate typically has 6, 12, 24, 96, 384 or even 1536 sample wells arranged in a rectangular matrix. Some microplates have even been manufactured with 3456 or even 9600 wells, and an “array tape” product has been developed that provides a continuous strip of microplates embossed on a flexible plastic tape.

Each well of a microplate typically holds somewhere between tens of nanolitres to several millilitres of liquid. The wells can be either circular or square. Microplates can be stored at low temperatures for long periods, may be heated to increase the rate of solvent evaporation from their wells and can even be heat-sealed with foil or clear film. Microplates with an embedded layer of filter material have been commercialized. Today there are microplates for just about every application, including, filtration, separation, optical detection, storage, reaction mixing or cell culturing.

In 1996, the Society for Biomolecular Sciences (SBS) began an initiative to create a standard definition of a microtiter plate. A series of standards was proposed in 2003 and published by the American National Standards Institute (ANSI) on behalf of the SBS. The standards govern various characteristics of a microplate including well dimensions (e.g. diameter, spacing and depth) as well as plate properties (e.g. dimensions and rigidity).

Published SBS references include: ANSI/SBS 1-2004: Microplates—Footprint Dimensions—last updated Jan. 9, 2004. ANSI/SBS 2-2004: Microplates—Height Dimensions—last updated Jan. 9, 2004. ANSI/SBS 3-2004: Microplates—Bottom Outside Flange Dimensions—last updated Jan. 9, 2004. ANSI/SBS 4-2004: Microplates—Well Positions—last updated Jan. 9, 2004. The published standards are incorporated herein by reference. Relevant sections of the SBS standards are reproduced below and in the figures. A number of companies have developed robots to specifically handle SBS microplates. These robots may include liquid handlers or pipettes which aspirate or dispense liquid samples from and to these plates, or “plate movers” which transport them between instruments, plate stackers which store microplates during these processes, plate hotels for longer term storage or microplate incubators to ensure constant environmental conditions during testing.

Some microorganisms tend to form a surface-layer of biomass on liquid growth media, in particular, filamentous fungi can sometimes form quite dense biomass mats on the surface of liquid growth media in microplate wells. It is important in automatic sampling systems to maintain accuracy and reproducibility in the sampling process and surface layers of biomass often hinder or clog the sampling needles of automated pipetting stations which leads to problems with reproducibility, reliability and process flow.

### SUMMARY OF THE INVENTION

A first aspect of the invention provides a microplate adapter for sampling a liquid growth medium from a microplate, where one or more microorganism has formed a layer of biomass on the surface of the liquid growth medium in one or more well, said adapter comprising a main body [FIG. 1: 1] with a plurality of hollow protruding fingers [FIG. 1: 3], wherein:
(a) each finger protrudes into a corresponding well of the microplate when the adapter is placed on top of the microplate, whereby said finger pushes any biomass layer on the surface down into the liquid growth medium;
(b) each finger has one or more openings in its bottom and/or sides to allow free flow of liquid from the well into the hollow of each finger, when the adapter is placed on top of the microplate and the surface layer of biomass is pushed down; and
(c) each finger has one or more openings at the top as well as internal dimensions that allow individual samples to be taken of the liquid in the hollow of each finger, when the adapter is placed on top of the microplate.

In a second aspect, the invention provides a method of sampling a liquid growth medium from a microplate, where one or more microorganism has formed a layer of biomass on the surface of the liquid growth medium in one or more well, said method comprising the steps of:
A) placing an adapter on top of the microplate, said adapter comprising a main body [FIG. 1: 1] with a plurality of hollow protruding fingers [FIG. 1: 3], wherein:
   (i) each finger protrudes into a corresponding well of the microplate when the adapter is placed on top of the microplate, whereby said finger pushes any biomass layer on the surface down into the liquid growth medium;
   (ii) each finger has one or more openings in its bottom and/or sides to allow free flow of liquid from the well into the hollow of each finger, when the adapter is placed on top of the microplate and the surface layer of biomass is pushed down; and
   (iii) each finger has one or more openings at the top as well as internal dimensions that allow individual samples to be taken of the liquid in the hollow of each finger, when the adapter is placed on top of the microplate; and
B) sampling the liquid growth medium from the hollow of at least one finger.

A third aspect of the invention relates to a method for transferring a surface-growing microorganisms from the surface of liquid medium in all wells of a microplate simultaneously, said method comprising:
A) placing an adapter on top of the microplate, said adapter comprising a main body [FIG. 1: 1] with a plurality of hollow protruding fingers [FIG. 1: 3], so that:
   (i) each finger protrudes into a corresponding well of the microplate;
   (ii) each finger has one or more openings in its bottom and/or sides to allow free flow of liquid from the well into and out of the hollow of each finger, when the adapter is placed on top of the microplate or removed from it; and
   (iii) each finger has one or more openings at the top to allow air to escape;
      wherein a liquid growth medium and a microorganism has been added to the wells of the microplate before, during or after step (A), but before step (B);
B) incubating the microplate and adapter together under conditions suitable for cultivating the microorganism, which then forms a layer of biomass on the surface of the liquid growth medium in the hollow of the protruding fingers;
C) removing the adapter from the microplate, so the liquid medium drains from the hollow of each finger while the surface-layer of biomass remains inside each finger, whereby the microorganisms from all the wells become transferable at once inside the fingers of the adapter, to be further processed or stored.

### BRIEF DESCRIPTION OF DRAWINGS

FIG. 1 shows transparent line-drawings of a sampling adapter according to the invention suitable for a standard 96-well SBS microplate viewed from the longest side (top drawing), from above (middle drawing) and from the short side (bottom drawing). The wells of microplates are typically filled about two thirds to the top with liquid. The adapter has 96 conical hollow protruding “fingers”, that each fit into one of the 96 wells of the microplate, when placed on top of the microplate. Each finger has a cross-shaped open cutout in the bottom, so that it will push any surface biomass-layer on the growth medium down into the medium in the wells, while allowing about half of the liquid medium of each well to flow into the hollow fingers, as the fingers of the adapter are inserted into the wells of the microplate when the adapter is placed on top of the microplate.

FIG. 2 also shows a transparent line-drawing of a sampling adapter according to the invention suitable for a standard 96-well SBS microtiter plate viewed from the longest side (top drawing), from above (middle drawing) and from the short side (bottom drawing). However, instead of a cross-shaped cutout in a closed-ended finger (as shown in FIG. 1), this adapter design features open-ended fingers with a cross-hair obsctruction to push down the biomass surface-layers.

FIG. 3 is identical to FIG. 2 but includes suggested suitable measurements in mm for manufacturing of the adapter in plastic, e.g., polypropylene or polystyrene, by injection-molding.

FIG. 4 shows two photos of a prototype sampling adapter according to the invention suitable for a standard 96-well SBS microtiter plate; viewed from above (top photo), from below (middle photo) and showing a close-up of the unit viewed from below (bottom photo). This prototype was made by cast milling.

FIG. 5 shows another transparent line-drawing of a sampling adapter according to the invention suitable for a standard 96-well SBS microtiter plate viewed from the longest side (top left drawing), the short side (top right drawing) and from above (lower drawing). This adapter is flat with minimal or no side flanges and instead of a cross-shaped cutout in the bottom of each closed-sided protruding finger (as shown in FIG. 1), each finger in this adapter has a round disc in the bottom and open slits in the sides to allow easy flow of growth medium into the hollow fingers. Suggested measurements in mm are indicated that are suitable for manufacturing of the adapter in plastic, e.g., polypropylen or polystyren, by injection-molding or stereolithography. Pictures of a prototype according to this drawing are shown in FIG. 18.

FIG. 6 shows a microplate footprint copied from ANSI/SBS 1-2004: 1) The drawing standard used is ASME Y14.5M-1994. 2) The geometry shown is for illustration only and does not imply any preferred or required construction. 3) Dimensions shown are: Millimeters/(Inches). 4) Dimensions and tolerances do not include draft. 5) The footprint must be continuous and uninterrupted around the base of the plate. A) A tolerance ±0.5 mm (0.0197 inches) applies overall, a tolerance of ±0.25 mm (0.0098 inches) applies at zones B-G, C-F, D-J & E-H.

FIG. 7 shows a mechanical drawing defining the height of a typical microplate: 1) The drawing standard used is ASME Y14.5M-1994. 2) The geometry shown is for illustration only and does not imply any preferred or required construction. 3) Dimensions shown are: Millimeters/(Inches). 4) Dimensions and tolerances do not include draft. A) Typical height=14.35 mm (0.56560 inches)±0.76 mm (0.0299 inches) applied overall, and a tolerance of ±0.25 mm (0.0098 inches) applied within area “K”. B) The 1mm (0.0394 inch) clearance applies in the area of the wells only.

FIG. 8 shows a mechanical drawing defining the flange dimensions of a microplate. 1) The drawing standard used is ASME Y14.5M-1994. 2) The geometry shown is for illustration only and does not imply any preferred or required construction. 3) Dimensions shown are: Millimeters/(Inches). 4) Dimensions and tolerances do not include draft. Note A)
SBS-3, 4.1 short flange height=2.41±0.38 mm (0.0948±0.0150 inches)
SBS-3, 4.2 medium flange height=6.10±0.38 mm (0.2402±0.0150 inches)
SBS-3, 4.3 tall flange height=7.62±0.38 mm (0.3000±0.0150 inches)
SBS-3, 4.4 short flange height with interruptions=2.41 ±0.38 mm (0.0948±0.0150 inches) projections allowed as shown.
SBS-3, 4.5 dual flange heights=2.41 ±0.38 mm (0.0948±0.0150 inches) at short sides, 7.62±0.38 mm (0.3000±0.0150 inches) at long sides.
   Note B) The flange height for SBS-3a, 3b, and 3c must be the same on all four sides. Note C) Quantity and location of chamfers(s) is optional. If used the chamfer must not include the flange.

FIG. 9 shows the well positions of a 96-well microplate. 1) The drawing standard used is ASME Y14.5M-1994. 2) The geometry shown is for illustration only and does not imply any preferred or required construction. 3) Dimensions shown are: Millimeters/(Inches). 4) Dimensions and tolerances do not include draft. A) The top left well of the plate shall be clearly marked (e.g.: on the left with the letter “A” or the numeral “1”, or at the top with the numeral “1”). Additional markings may be provided.

FIG. 10 shows the well positions of a 384-well microplate. 1) The drawing standard used is ASME Y14.5M-1994. 2) The geometry shown is for illustration only and does not imply any preferred or required construction. 3) Dimensions shown are: Millimeters/(Inches). 4) Dimensions and tolerances do not include draft. A) The top left well of the plate shall be clearly marked (e.g.: on the left with the letter “A” or the numeral “1”, or at the top with the numeral “1”). Additional markings may be provided.

FIG. 11 shows the well positions of a 1536-well microplate. 1) The drawing standard used is ASME Y14.5M-1994. 2) The geometry shown is for illustration only and does not imply any preferred or required construction. 3) Dimensions shown are: Millimeters/(Inches). 4) Dimensions and tolerances do not include draft. A) The top left well of the plate shall be clearly marked (e.g.: on the left with the letter “A” or the numeral “1”, or at the top with the numeral “1”). Additional markings may be provided.

FIG. 12 shows drawings and dimensions of the NUNC™ microplate Multidish 6.

FIG. 13 shows drawings and dimensions of the NUNC™ microplate Multidish 12.

FIG. 14 shows drawings and dimensions of the NUNC™ microplate Multidish 24.

FIG. 15 shows drawings and dimensions of the NUNC™ microplate Multidish 48.

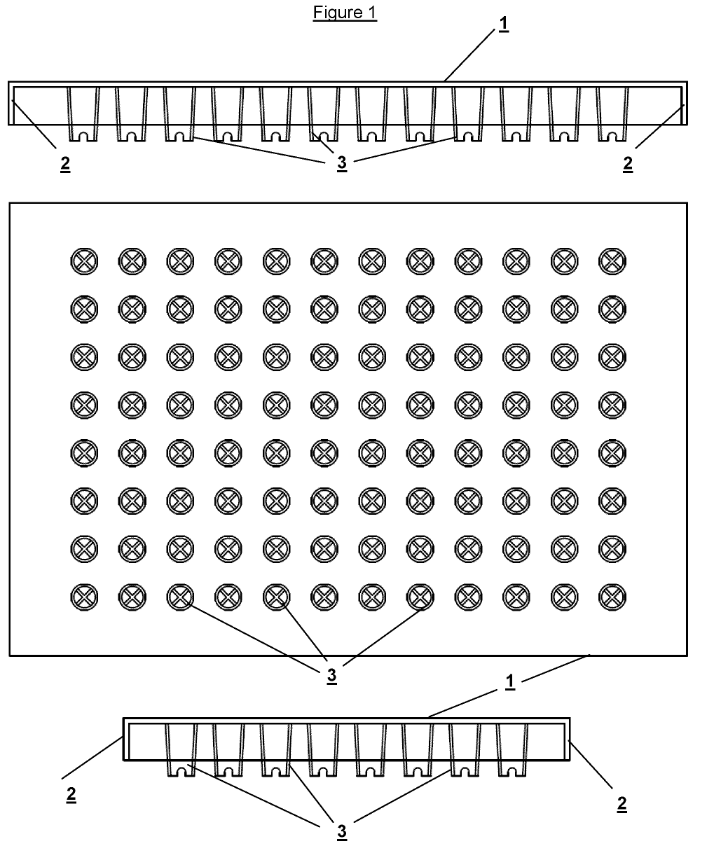

FIG. 16 shows another transparent line-drawing of a sampling adapter according to the invention suitable for a standard 96-well SBS microtiter plate viewed from above (top drawing), longest side (middle drawing) and the short side (lower drawing). This adapter is similar to that of FIG. 5; flat with minimal or no side flanges and instead of a cross-shaped cutout in the bottom of each closed-sided protruding finger (as shown in FIG. 1), each finger in this adapter has a round hole in the bottom and open slits in the sides to allow easy flow of growth medium into the hollow fingers. In addition, eight small bumps are included on the underside of the adapter to ensure some space between the adapter and the microplate, when the adapter is placed on the microplate. Suggested measurements in mm are indicated that are suitable for manufacturing of the adapter in plastic, e.g., polypropylen or polystyren, by injection-molding.

FIG. 17 shows pictures of a steel prototype sampling adapter according to the invention suitable for a standard 24-well microtiter plate viewed from above (top photo), longest side (middle photo) and from below, where the fingers each have a wire-mesh opening (lower photo).

FIG. 18 shows photos of a polymer prototype made in accordance with the schematics in FIG. 5 by stereolithography. The top photo shows the adapter from above, the middle from the side and the lower photo from the bottom.

### DETAILED DESCRIPTION OF THE INVENTION

The first aspect of the invention relates to a microplate adapter for sampling a liquid growth medium from a microplate, where one or more microorganism has formed a layer of biomass on the surface of the liquid growth medium in one or more well, said adapter comprising a main body [FIG. 1: 1] with a plurality of hollow protruding fingers [FIG. 1: 3], wherein:
(a) each finger protrudes into a corresponding well of the microplate when the adapter is placed on top of the microplate, whereby said finger pushes any biomass layer on the surface down into the liquid growth medium;
(b) each finger has one or more openings in its bottom and/or sides to allow free flow of liquid from the well into the hollow of each finger, when the adapter is placed on top of the microplate and the surface layer of biomass is pushed down; and
(c) each finger has one or more openings at the top as well as internal dimensions that allow individual samples to be taken of the liquid in the hollow of each finger, when the adapter is placed on top of the microplate.

The second aspect of the invention relates to a method of sampling a liquid growth medium from a microplate, where one or more microorganism has formed a layer of biomass on the surface of the liquid growth medium in one or more well, said method comprising the steps of:
A) placing an adapter on top of the microplate, said adapter comprising a main body [FIG. 1: 1] with a plurality of hollow protruding fingers [FIG. 1: 3], wherein:
   (i) each finger protrudes into a corresponding well of the microplate when the adapter is placed on top of the microplate, whereby said finger pushes any biomass layer on the surface down into the liquid growth medium;
   (ii) each finger has one or more openings in its bottom and/or sides to allow free flow of liquid from the well into the hollow of each finger, when the adapter is placed on top of the microplate and the surface layer of biomass is pushed down; and
   (iii) each finger has one or more openings at the top as well as internal dimensions that allow individual samples to be taken of the liquid in the hollow of each finger, when the adapter is placed on top of the microplate; and
B) sampling the liquid growth medium from the hollow of at least one finger.

In a third aspect, the invention relates to a method for transferring a surface-growing microorganism from the surface of liquid medium in all wells of a microplate simultaneously, said method comprising:
A) placing an adapter on top of the microplate, said adapter comprising a main body [FIG. 1: 1] with a plurality of hollow protruding fingers [FIG. 1: 3], so that:
   (i) each finger protrudes into a corresponding well of the microplate;
   (ii) each finger has one or more openings in its bottom and/or sides to allow free flow of liquid from the well into and out of the hollow of each finger, when the adapter is placed on top of the microplate or removed from it; and
   (iii) each finger has one or more openings at the top to allow air to escape;
      wherein a liquid growth medium and a microorganism has been added to the wells of the microplate before, during or after step (A), but before step (B);
B) incubating the microplate and adapter together under conditions suitable for cultivating the microorganism, which then forms a layer of biomass on the surface of the liquid growth medium in the hollow of the protruding fingers;
C) removing the adapter from the microplate, so the liquid medium drains from the hollow of each finger while the surface-layer of biomass remains inside each finger, whereby the microorganisms from all the wells become transferable at once inside the fingers of the adapter, to be further processed or stored.

A preferred embodiment of the third aspect of the invention comprises an additional step D) sampling the spent liquid growth medium in one or more wells of the microplate.

Preferably in all aspect and embodiments of the invention, the adapter is disposable, i.e., made from a polymer material.

In preferred embodiments of all aspects of the invention, the microplate adapter is suitable for a 6, 12, 24, 48, 96, 384 or 1536 well microplate; preferably the microplate meets the Standards ANSI/SBS 1-2004 through ANSI/SBS 4-2004.

Preferably, the protruding fingers [FIG. 1: 3] extend anywhere from at least 1 mm into the corresponding wells, when the adapter is placed on top of the microplate; preferably the fingers extend at least 2, 3, 4, 5, 6, 7, 8, 9, 10, 11, 12, 13 or even 14 mm into the corresponding wells, when the adapter is placed on top of the microplate

It is also preferred, that the protruding fingers [FIG. 1: 3] are tapered or conical on the outside, thereby allowing easier insertion of the fingers into the wells of the microplate; preferably the sides are angled between 0.1 and 20 degrees.

In another preferred embodiment the protruding fingers [FIG. 1: 3] are tapered or conical on the inside, thereby allowing easier sampling or insertion of a disposable pipette tip for sampling; preferably the sides are angled between 0.1 and 20 degrees.

The microplate adapter may have one or more sides that keep the adapter more securely in place once is has been fitted to the microplate. Preferably, the microplate adapter has one or more sides that fit around the outside of the microplate; preferably the sides are at least 1 mm high, more preferably at least 2, 3, 4, 5, 6, 7, 8, 9, 10 mm high.

Microplates often have one or more chamfers at the corner of the plate to ensure its proper orientation. Accordingly, it is preferred that the microplate adapter of the invention has one or more chamfer corresponding to any chamfer on a standard microplate which meets the Standards ANSI/SBS 1-2004 through ANSI/SBS 4-2004.

### Adapter Footprint

With reference to the figures, the adapter of the invention generally comprises a main body [FIG. 1: 1]. In some embodiments, the main body is substantially planar [FIG. 5]. In some embodiments, the adapter comprises an optional skirt or flange portion [FIG. 1: 2] disposed about a periphery of main body. The skirt of flange portion can form a lip around the main body and can vary in height. The skirt or flange portion can facilitate correct alignment or placement of the adapter on a microplate. Additionally, the skirt or flange portion can provide additional rigidity to the adapter such that during handling, insertion, and the like, the adapter remains rigid. However, in some embodiments, the adapter can employ a skirtless design (see FIG. 5) to reduce cost of materials for construction and/or to allow the fitting of a standard microplate lid on top of the adapter after its placement on a microplate, depending upon user preference.

In order to facilitate use with existing equipment, robotic implements, and instrumentation, the footprint dimensions of the main body [FIG. 1: 1] of the adapter and/or skirt/flange portion of the adapter [FIG. 1: 2], in some embodiments, can conform to published standards specified by the Society of Biomolecular Screening (SBS) and the American National Standards Institute (ANSI).

### Adapter Protrusions/Fingers

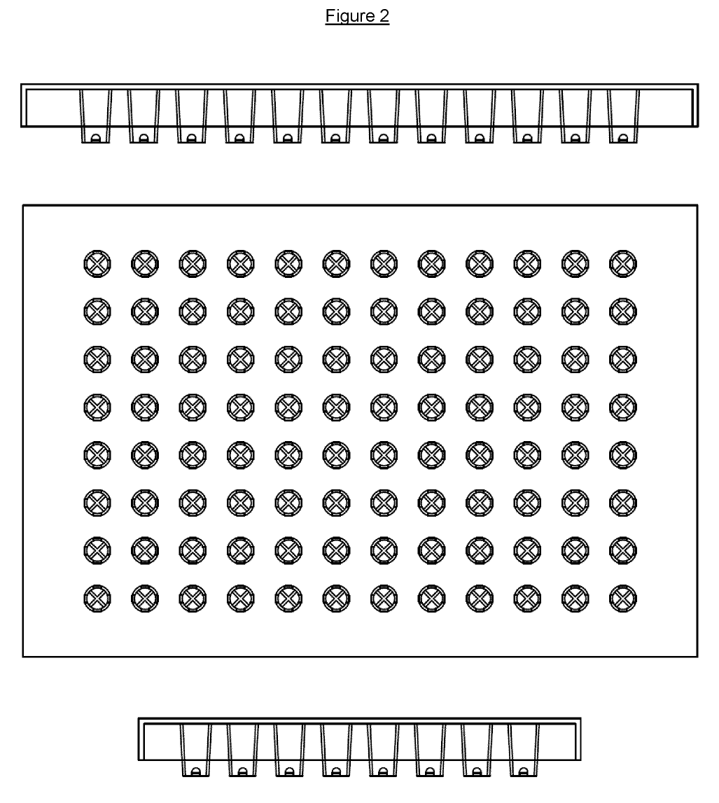

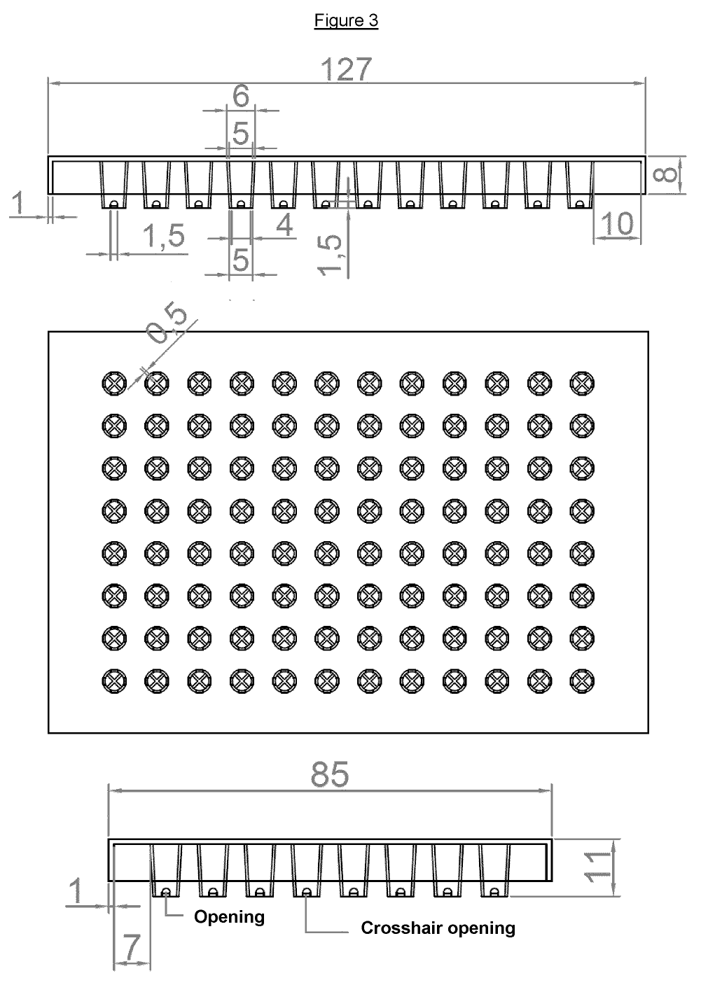

According to some embodiments, as illustrated in the Figures, the main body of the adapter comprises a plurality of hollow and partially open protruding fingers [FIG. 1: 3] that can be substantially equivalent in size. The plurality of fingers can have any cross-sectional shape. In some embodiments, as illustrated in the Figures, each of the plurality of fingers comprises a generally circular rim portion with a downwardly-extending, generally-continuous sidewall that terminates with an open bottom or to a bottom interconnected to the sidewall with an outside radius that needs to be smaller than the inside radius of the wells of the microplate to which the adapter is intended. A draft angle of the sidewall can be used in some embodiments to make the finger conical (see FIGS. 1-5). In some embodiments, the draft angle provides benefits including increased ease of manufacturing and ease of insertion into the microplate wells. The particular draft angle is determined, at least in part, by the manufacturing method and the size of each of the plurality of wells of the microplate to which the adapter is intended. In some embodiments, the draft angle of the sidewall of each finger can be about 1° to 5° or greater.

According to some embodiments, each of the plurality of fingers comprises a generally square-shaped rim portion with downwardly-extending sidewalls that terminate with an open bottom or to a bottom interconnected to the sidewall with an outside radius that needs to be smaller than the inside radius of the wells of the microplate to which the adapter is intended. A draft angle of the sidewall can be used in some embodiments to make the finger conical (see FIGS. 1-5). In some embodiments, the draft angle provides benefits including increased ease of manufacturing and ease of insertion into the microplate wells. The particular draft angle is determined, at least in part, by the manufacturing method and the size of each of the plurality of wells of the microplate to which the adapter is intended. In some embodiments, the draft angle of the sidewall of each finger can be about 1° to 5° or greater.

In some embodiments, and in some configurations, the plurality of fingers comprising a generally circular rim portion can provide advantages over the fingers comprising a generally square-shaped rim portion. During manufacture of the adapter, in some cases cylindrically or conically shaped mold pins used to form the plurality of fingers comprising a generally circular rim portion can permit unencumbered flow of molten polymer.

In some embodiments, the adapter of the invention comprises an alignment feature, such as a corner chamfer, a pin, a slot, a cut corner, an indentation, a graphic, or other unique feature that is capable of interfacing with a corresponding feature formed in the microplate for which the adapter is intended. In some embodiments, the alignment feature comprises a nub or protrusion.

In order to facilitate use with existing equipment, robotic implements, and instrumentation and to ensure proper insertion of the adapter fingers into the wells of microplates, the outside dimensions and placements of the adapter fingers may conform to published standards for internal dimensions and positioning of wells in microplates as specified by the Society of Biomolecular Screening (SBS) and the American National Standards Institute (ANSI) mentioned elsewhere herein. In particular, all the fingers' outside dimensions should be at least 1 mm smaller than those of the wells in order to allow insertion into the wells.

### Adapter Molding

In some embodiments, the adapter can be molded by first extruding a melt blend comprising a mixture of a polymer and one or more thermally conductive materials and/or additives. In some embodiments, the polymer and thermally conductive additives can be fed into a twin-screw extruder using a gravimetric feeder to create a well-dispersed melt blend. In some embodiments, the extruded melt blend can be transferred through a water bath to cool the melt blend before being pelletized and dried. The pelletized melt blend can then be heated above its melting point by an injection molding machine and then injected into a mold cavity. The mold cavity can generally conform to a desired shape of the adapter. In some embodiments, the injection-molding machine can cool the injected melt blend to create the adapter. Finally, the adapter can be removed from the injection-molding machine.

In some embodiments, two or more material types of pellets can be mixed together and the combination then placed in the injection molding machine to be melt blended during the injection molding process. In some embodiments, the adapter can be molded by first receiving pellet material from a resin supplier; drying the pellet material in a resin dryer; transferring the dried pellet material with a vacuum system into a hopper of a mold press; molding the adapter; trimming any resultant gates or flash; and packaging the adapter.

Referring to the Figures, in some embodiments, the main body of the adapter is formed to include the plurality of through holes extending therethrough—that is, the plurality of through holes are enclosed at a bottom thereof. In such arrangements, a backing can be coupled to the main body to, at least in part, seal or otherwise enclose the plurality of through holes to form the plurality of protruding fingers. However, the main body can comprise a self-contained, self-defined plurality of protruding fingers in that a bottom structure is formed to prevent a through-hole configuration.

The backing can be coupled to the main body, in some embodiments, through the use of insert molding. In insert molding, the backing, which can comprise either a single material or a laminate as described herein, can be placed within a mold cavity prior to injection of molten material. Upon injection of molten molding material into the mold cavity, melting and later bonding of the injected material with the material of the backing can be completed.

In some embodiments, the backing can be coupled to main body through laser welding. In such arrangement, a laser source can be used to emit a laser beam. Depending upon the particular configuration of the backing, the laser source can be positioned either above or below main body and backing and the materials thereof can be selected to permit the laser beam to enter one of the main body and backing and pass to a weld zone. For example, if the laser source is positioned above main body and backing, the material of main body can be selected to be transmissive to the laser beam while the material of backing can be absorptive to the laser beam. As the laser beam passes through main body it impacts backing. As a result of the backing being made of an absorptive material, the backing is heated to a melting point of the main body and/or backing along a weld zone between the main body and the backing. The resultant molten material near the weld zone then bonds or otherwise fuses to cause the main body and the backing to be welded together once cooled below the melting point. In some embodiments, the backing can be coupled to the main body through ultrasonic welding, film decorating-type processing within the injection mold, or similar processes.

Partial reproduction of ANSI/SBS 1-2004: Microplate footprint dimensions There is one annex A in this standard which is not included herein. Annex A is informative and not considered part of the standard. It is provided as an aid only for the interpretation of specific elements of ASME Y14.5 as they apply to figures in SBS standards.

### 1. Scope and Purpose

### 1.1 Scope:

This standard defines the dimensional requirements of the footprint of a microplate as specified in American National Standards covering these microplates.

### 1.2 Purpose:

It is the purpose of this standard to describe the minimal dimensions required of a microplate that is considered to meet the standards. This standard also outlines the conditions required for making necessary measurements. Unless otherwise specified, all dimensions are applicable at 20 degrees C. (68 degrees F.). Compensation may be made for measurements made at other temperatures.

### 2. Normative References

The following standards contain provisions which, through reference in this text, constitute provisions of this American National Standard. At the time of publication, the editions indicated were valid. All standards are subject to revision, and parties to agreements based on this American National Standard are encouraged to investigate the possibility of applying the most recent editions of the standards listed below. ASME Y14.5M-1994, Dimensioning and Tolerancing

### 3. Definitions

There are many terms and definitions associated with microplates that have special meaning to the industry. The following are definitions of terms used in this document:
3.1 ASME: Abbreviation for the American Society of Mechanical Engineers
3.2 ANSI: Abbreviation for the American National Standards Institute, Inc.
3.3 SBS: Abbreviation for the Society for Biomolecular Screening.
   4. Microplate footprint
   4.1 Normal tolerances

### 4.1.1 Footprint

4.1.1.1 The outside dimension of the base footprint, measured within 12.7 mm (0.5000 inches) of the outside corners, shall be as follows:
Length 127.76 mm±0.25 mm (5.0299 inches±0.0098 inches)
Width 85.48 mm±0.25 mm (3.3654 inches±0.0098 inches)
   4.1.1.2 The outside dimension of the base footprint, measured at any point along the side, shall be as follows:
Length 127.76 mm±0.5 mm (5.0299 inches±0.0197 inches)
Width 85.48 mm±0.5 mm (3.3654 inches±0.0197 inches)
   4.1.1.3 The footprint must be continuos and uninterrupted around the base of the plate.

### 4.1.2 Corner Radius

4.1.2.1 The four outside corners of the plate's bottom flange shall have a corner radius to the outside of 3.18 mm±1.6 mm (0.1252 inch±0.0630 inches)

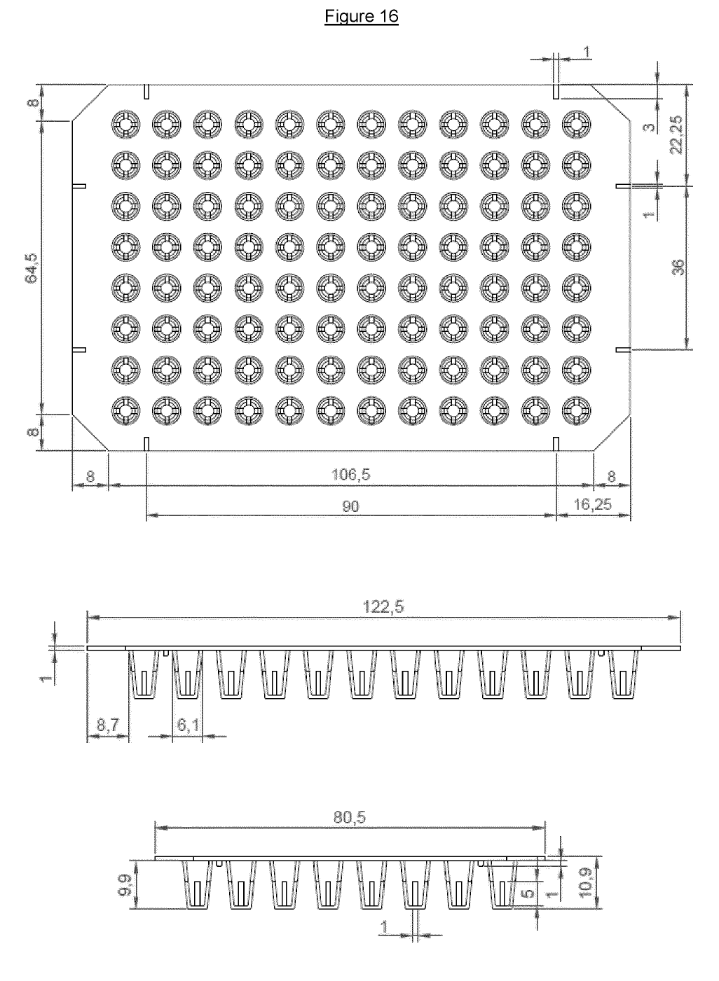

An explanatory figure is provided in FIG. 6 herein and further explanatory figures A.1-A.5 are available from Annex A of the publication ANSI/SBS 1-2004; they are incorporated herein in their entirety by reference.

### Partial Reproduction of ANSI/SBS 2-2004: Microplate Height Dimensions

There is one annex A in the standard which is not included herein. Annex A is informative and not considered part of this standard. It is provided as an aid only for the interpretation of specific elements of ASME Y14.5 as they apply to figures in SBS standards.

1. Scope and purpose

### 1.1 Scope:

This standard defines the dimensional requirements of the height of a microplate as specified in American National Standards covering these microplates.

### 1.2 Purpose:

It is the purpose of this standard to describe the minimal dimensions required of a microplate that is considered to meet the standards. This standard also outlines the conditions required for making necessary measurements. Unless otherwise specified, all dimensions are applicable at 20 degrees C. (68 degrees F.). Compensation may be made for measurements made at other temperatures.

### 2. Normative References

The following standards contain provisions which, through reference in this text, constitute provisions of this American National Standard. At the time of publication, the editions indicated were valid. All standards are subject to revision, and parties to agreements based on this American National Standard are encouraged to investigate the possibility of applying the most recent editions of the standards listed below. ASME Y14.5M-1994, Dimensioning and Tolerancing

### 3. Definitions

There are many terms and definitions associated with microplates that have special meaning to the industry. The following are definitions of terms used in this document:
3.1 ASME: Abbreviation for the American Society of Mechanical Engineers
3.2 ANSI: Abbreviation for the American National Standards Institute, Inc.
3.3 SBS: Abbreviation for the Society for Biomolecular Screening.

### 4 Microplate Height

Microplates that meet this standard may either comply with those standards specified in parts 4.1, or 4.2. Microplates, or instruments that use them, that advertise compliance with this standard must clearly state which of these two parts they meet.

4.1 Typical height with clearance

4.1.1 Plate height
4.1.1.1 The plate height, measured from Datum A (the resting plane) to the maximum protrusion of the perimeter wells, shall be 14.35 mm±0.25 mm (0.5650 inches±0.0098 inches)
4.1.1.2 The overall plate height, measured from Datum A (the resting plane) to the maximum protrusion of the plate, shall be 14.35 mm±0.76 mm (0.5650 inches±0.0299 inches)

4.1.2 Top Surface
4.1.2.1 The maximum allowable projection above the top-stacking surface is 0.76 mm (0.0299 inches). The top-stacking surface is defined as that surface on which another plate would rest when stacked one on another.
4.1.2.2 When resting on a flat surface, the top surface of the plate must be parallel to the resting surface within 0.76 mm (0.0299 inches)

4.1.3 External Clearance to the Plate Bottom

The minimum clearance from Datum A (the resting plane) to the plane of the bottom external surface of the wells shall be 1 mm (0.0394 inches). This clearance is limited to the area of the wells.

4.2 Typical height

4.2.1 Plate height
4.2.1.1 The plate height, measured from Datum A (the resting plane) to the maximum protrusion of the perimeter wells, shall be 14.35 mm±0.25 mm (0.5650 inches±0.0098 inches)
4.2.1.2 The overall plate height, measured from Datum A (the resting plane) to the maximum protrusion of the plate, shall be 14.35 mm±0.76 mm (0.5650 inches±0.0299 inches)

4.2.2 Top Surface
4.2.2.1 The maximum allowable projection above the top-stacking surface is 0.76 mm (0.0299 inches). The top-stacking surface is defined as that surface on which another plate would rest when stacked one on another.
4.2.2.2 When resting on a flat surface, the top surface of the plate must be parallel to the resting surface within 0.76 mm (0.0299 inches).

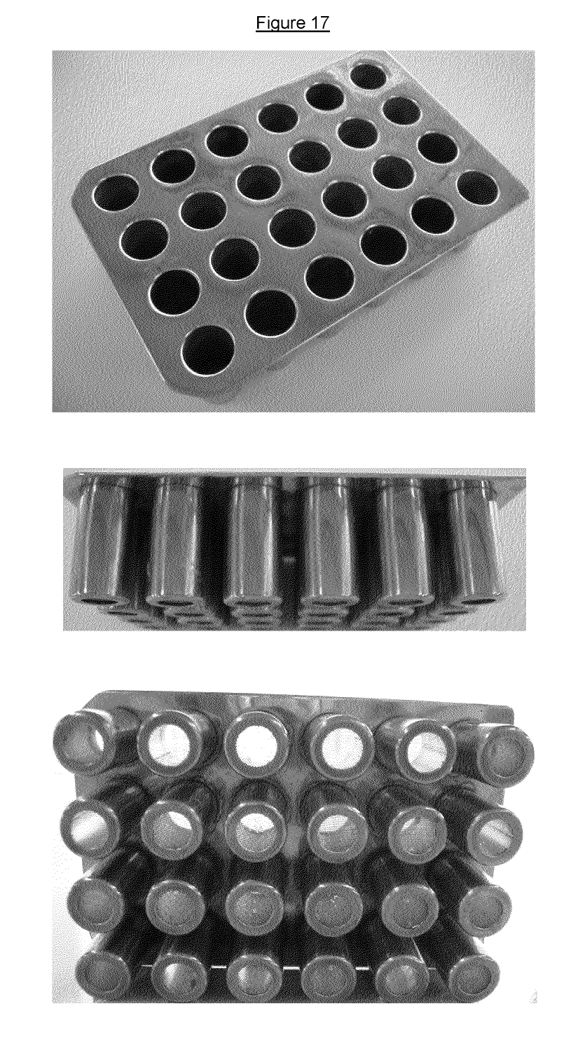

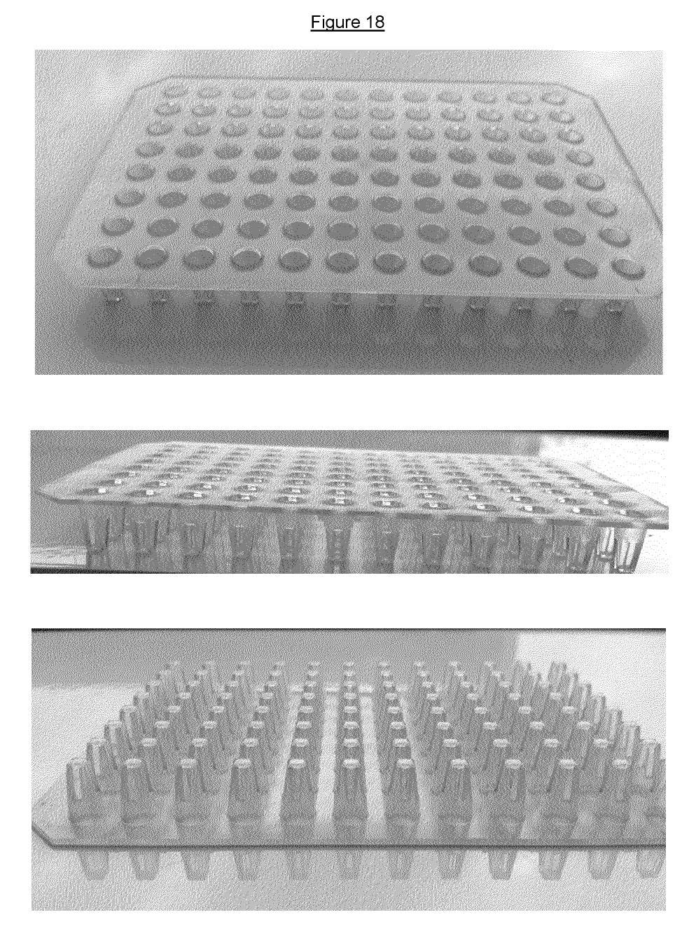

An explanatory figure is provided in FIG. 7 herein and further explanatory figures A.1-A.5 are available from Annex A of the publication ANSI/SBS 2-2004; they are incorporated herein in their entirety by reference.

### Partial Reproduction of ANSI/SBS 3-2004: Microplate Bottom Outside Flange Dimensions

There is one annex A in the standard which is not included herein. Annex A is informative and not considered part of this standard. It is provided as an aid only for the interpretation of specific elements of ASME Y14.5 as they apply to figures in SBS standards.

1. Scope and purpose

1.1 Scope:

This standard defines the dimensional requirements of the bottom outside flange of a microplate as specified in American National Standards covering these microplates.

1.2 Purpose:

It is the purpose of this standard to describe the minimal dimensions required of a microplate that is considered to meet the standards. This standard also outlines the conditions required for making necessary measurements. Unless otherwise specified, all dimensions are applicable at 20 degrees C. (68 degrees F.). Compensation may be made for measurements made at other temperatures.

2. Normative references

The following standards contain provisions which, through reference in this text, constitute provisions of this American National Standard. At the time of publication, the editions indicated were valid. All standards are subject to revision, and parties to agreements based on this American National Standard are encouraged to investigate the possibility of applying the most recent editions of the standards listed below. ASME Y14.5M-1994, Dimensioning and Tolerancing.

### 3. Definitions

There are many terms and definitions associated with microplates that have special meaning to the industry. The following are definitions of terms used in this document:
3.1 ASME: Abbreviation for the American Society of Mechanical Engineers
3.2 ANSI: Abbreviation for the American National Standards Institute, Inc.
3.3 SBS: Abbreviation for the Society for Biomolecular Screening.
   4. Bottom-outside flange

Microplates that meet this standard may either comply with those standards specified in parts 4.1, 4.2, 4.3, 4.4, or 4.5. Microplates, or instruments that use them, that advertise compliance with this standard must clearly state which of these five parts they meet.

4.1 Short flange height

4.1.1 Flange height
4.1.1.1 The height of the bottom outside flange shall be 2.41 mm±0.38 mm (0.0948 inches±0.0150 inches). This is measured from Datum A (the bottom-resting plane) to the top of the flange.
4.1.1.2 All four sides must have the same flange height.

4.1.2 Flange width
4.1.2.1 The width of the bottom outside flange measured at the top of the flange shall be a minimum of 1.27 mm (0.0500 inches).

4.1.3 Chamfers (Corner Notches)
4.1.3.1 The quantity and location of chamfer(s) is optional. If used, the chamfer must not include the flange.
   4.2 Medium flange height

4.2.1 Flange height
4.2.1.1 The height of the bottom outside flange shall be 6.10 mm±0.38 mm (0.2402 inches±0.0150 inches). This is measured from Datum A (the bottom-resting plane) to the top of the flange.
4.2.1.2 All four sides must have the same flange height.

4.2.2 Flange width
4.2.2.1 The width of the bottom outside flange measured at the top of the flange shall be a minimum of 1.27 mm (0.0500 inches).

4.2.3 Chamfers (Corner Notches)
4.2.3.1 The quantity and location of chamfer(s) is optional. If used, the chamfer must not include the flange.

### 4.3 Tall Flange Height

4.3.1 Flange height
4.3.1.1 The height of the bottom outside flange shall be 7.62 mm±0.38 mm (0.3000 inches±0.0150 inches). This is measured from Datum A (the bottom-resting plane) to the top of the flange.
4.3.1.2 All four sides must have the same flange height.

4.3.2 Flange width
4.3.2.1 The width of the bottom outside flange measured at the top of the flange shall be a minimum of 1.27 mm (0.0500 inches).

4.3.3 Chamfers (Corner Notches)
4.3.3.1 The quantity and location of chamfer(s) is optional. If used, the chamfer must not include the flange.
   4.4 Short flange height with interruptions

4.4.1 Flange height
4.4.1.1 The height of the bottom outside flange shall be 2.41 mm±0.38 mm (0.0948 inches±0.0150 inches). This is measured from Datum A (the bottom-resting plane) to the top of the flange.
4.4.1.2 All four sides must have the same flange height except for an allowable interruption centered along the long side.

4.4.2 Interruptions
4.4.2.1 Each of the long sides of the plate shall be allowed to have a single interruption or projection on center.
4.4.2.2 Each edge of the interruption shall be a minimum of 47.8 mm (1.8819 inches) from the nearest edge of the part.
4.4.2.3 The height of the flange at the interruption shall not exceed 6.85 mm (0.2697 inches)

4.4.3 Flange width
4.4.3.1 The width of the bottom outside flange measured at the top of the flange shall be a minimum of 1.27 mm (0.0500 inches).4.4.4 Chamfers (Corner Notches).
4.4.4.1 The quantity and location of chamfer(s) is optional. If used, the chamfer must not include the flange.
   4.5 Dual flange heights

4.5.1 Flange height
4.5.1.1 The height of the bottom outside flange shall be 2.41 mm±0.38 mm (0.0948 inches±0.0150 inches) along the short sides of the plate. This is measured from Datum A (the bottom-resting plane) to the top of the flange.
4.5.1.2 The height of the bottom outside flange shall be 7.62 mm±0.38 mm (0.3000 inches±0.0150 inches) along the long sides of the plate. This is measured from Datum A (the bottom-resting plane) to the top of the flange.

4.5.2 Flange width
4.5.2.1 The width of the bottom outside flange measured at the top of the flange shall be a minimum of 1.27 mm (0.0500 inches).

4.5.3 Chamfers (Corner Notches)
4.5.3.1 The quantity and location of chamfer(s) is optional. If used, the chamfer must not include the flange.

An explanatory figure is provided in FIG. 8 herein and further explanatory figures A.1-A.5 are available from Annex A of the publication ANSI/SBS 3-2004; they are incorporated herein in their entirety by reference.

### Partial Reproduction of ANSI/SBS 4-2004: Microplate Well Positions

There is one annex A in the standard which is not included herein. Annex A is informative and not considered part of this standard. It is provided as an aid only for the interpretation of specific elements of ASME Y14.5 as they apply to figures in SBS standards.

1. Scope and purpose

1.1 Scope:

This standard defines the well center positional requirements of a microplate as specified in American National Standards covering these microplates.

1.2 Purpose:

It is the purpose of this standard to describe the minimal dimensions required of a microplate that is considered to meet the standards. This standard also outlines the conditions required for making necessary measurements. Unless otherwise specified, all dimensions are applicable at 20 degrees C. (68 degrees F.). Compensation may be made for measurements made at other temperatures.

### 2. Normative References

The following standards contain provisions which, through reference in this text, constitute provisions of this American National Standard. At the time of publication, the editions indicated were valid. All standards are subject to revision, and parties to agreements based on this American National Standard are encouraged to investigate the possibility of applying the most recent editions of the standards listed below. ASME Y14.5M-1994, Dimensioning and Tolerancing.

### 3. Definitions

There are many terms and definitions associated with microplates that have special meaning to the industry. The following are definitions of terms used in this document:
3.1 ASME: Abbreviation for the American Society of Mechanical Engineers
3.2 ANSI: Abbreviation for the American National Standards Institute, Inc.
3.3 SBS: Abbreviation for the Society for Biomolecular Screening.

### 4. Well Positions

Microplates that meet this standard may either comply with those standards specified in parts 4.1, 4.2, or 4.3. Microplates, or instruments that use them, that advertise compliance with this standard must clearly state which of these three parts they meet.

4.1 96 well microplate

4.1.1 Well layout
4.1.1.1 The wells in a 96 well microplate should be arranged as eight rows by twelve columns.

4.1.2 Well column position
4.1.2.1 The distance between the left outside edge of the plate and the center of the first column of wells shall be 14.38 mm (0.5661 inches)
4.1.2.2 The left edge of the part will be defined as the two 12.7 mm areas (as measured from the corners) as specified in SBS-1
4.1.2.3 Each following column shall be an additional 9. mm (0.3543 inches) in distance from the left outside edge of the plate.

4.1.3 Well row position
4.1.3.1 The distance between the top outside edge of the plate and the center of the first row of wells shall be 11.24 mm (0.4425 inches)
4.1.3.2 The top edge of the part will be defined as the two 12.7 mm areas (as measured from the corners) as specified in SBS-1
4.1.3.3 Each following row shall be an additional 9. mm (0.3543 inches) in distance from the top outside edge of the plate.

4.1.4 Positional Tolerance
4.1.4.1 The positional tolerance of the well centers will be specified using so called “True Position”. The center of each well will be within a 0.70 mm (0.0276 inches) diameter of the specified location. This tolerance will apply at “RFS” (regardless of feature size).

4.1.5 Well Markings
4.1.5.1 The top left well of the plate shall be marked in a distinguishing manner. Such distinguishing marks include, but are not limited to the following:
The top left well of the plate can be marked with the letter A or numeral 1 located on the left-hand side of the well.
The top left well of the plate can be marked with a numeral 1 located on the upper side of the well.
4.1.5.2 Additional markings may be provided.
   4.2 384 well microplate

4.2.1 Well layout
4.2.1.1 The wells in a 384 well microplate should be arranged as sixteen rows by twenty-four columns.

4.2.2 Well column position
4.2.2.1 The distance between the left outside edge of the plate and the center of the first column of wells shall be 12.13 mm (0.4776 inches)
4.2.2.2 The left edge of the part will be defined as the two 12.7 mm areas (as measured from the corners) as specified in SBS-1
4.2.2.3 Each following column shall be an additional 4.5 mm (0.1772 inches) in distance from the left outside edge of the plate.

4.2.3 Well row position
4.2.3.1 The distance between the top outside edge of the plate and the center of the first row of wells shall be 8.99 mm (0.3539 inches)
4.2.3.2 The top edge of the part will be defined as the two 12.7 mm areas (as measured from the corners) as specified in SBS-1
4.2.3.3 Each following row shall be an additional 4.5 mm (0.1772 inches) in distance from the top outside edge of the plate.

4.2.4 Positional Tolerance
4.2.4.1 The positional tolerance of the well centers will be specified using so called “True Position”. The center of each well will be within a 0.70 mm (0.0276 inches) diameter of the specified location. This tolerance will apply at “RFS” (regardless of feature size).

4.2.5 Well Markings
4.2.5.1 The top left well of the plate shall be marked in a distinguishing manner. Such distinguishing marks include, but are not limited to the following:
The top left well of the plate can be marked with the letter A or numeral 1 located on the left-hand side of the well.
The top left well of the plate can be marked with a numeral 1 located on the upper side of the well.
4.2.5.2 Additional markings may be provided.
   4.3 1536 well microplate

4.3.1 Well layout
4.3.1.1 The wells in a 1536 well microplate should be arranged as thirty-two rows by forty-eight columns.

4.3.2 Well column position
4.3.2.1 The distance between the left outside edge of the plate and the center of the first column of wells shall be 11.005 mm (0.4333 inches)
4.3.2.2 The left edge of the part will be defined as the two 12.7 mm areas (as measured from the corners) as specified in SBS-1
4.3.2.3 Each following column shall be an additional 2.25 mm (0.0886 inches) in distance from the left outside edge of the plate.

4.3.3 Well row position
4.3.3.1 The distance between the top outside edge of the plate and the center of the first row of wells shall be 7.865 mm (0.3096 inches)
4.3.3.2 The top edge of the part will be defined as the two 12.7 mm areas (as measured from the corners) as specified in SBS-1
4.3.3.3 Each following row shall be an additional 2.25 mm (0.0886 inches) in distance from the top outside edge of the plate.

4.3.4 Positional Tolerance
4.3.4.1 The positional tolerance of the well centers will be specified using so called “True Position”. The center of each well will be within a 0.50 mm (0.0197 inches) diameter of the specified location. This tolerance will apply at “RFS” (regardless of feature size).

4.3.5 Well Markings
4.3.5.1 The top left well of the plate shall be marked in a distinguishing manner. Such distinguishing marks include, but are not limited to the following:
The top left well of the plate can be marked with the letter A or numeral 1 located on the left-hand side of the well.
The top left well of the plate can be marked with a numeral 1 located on the upper side of the well.

4.3.5.2 Additional markings may be provided.

Explanatory figures are provided in FIGS. 9-11 herein and further explanatory figures A.1-A.6 are available from Annex A of the publication ANSI/SBS 4-2004; they are incorporated herein in their entirety by reference.

### EXAMPLES

### Example 1

### Adapter-Enabled Sampling from Microtiter Plate with Aspergillus oryzae

MDU-2BP medium: per liter 45 g maltose-1H₂O, 7 g yeast extract, 12 g KH₂ PO₄, 1 g MgSO₄-7H₂O, 2 g K₂SO₄, 5 g Urea, 1 g NaCl, 0.5 ml AMG trace metal solution, pH 5.0.

The filamentous fungus Aspergillus oryzae was grown in a standard SBS microtiter plate (96-well) for 4 days at 30° C. in MDU-2BP broth (200 microliter/well). After incubation fungal growth was observed on the surface of the growth medium in the wells forming a dense mat of organic material on the media surface in each well.

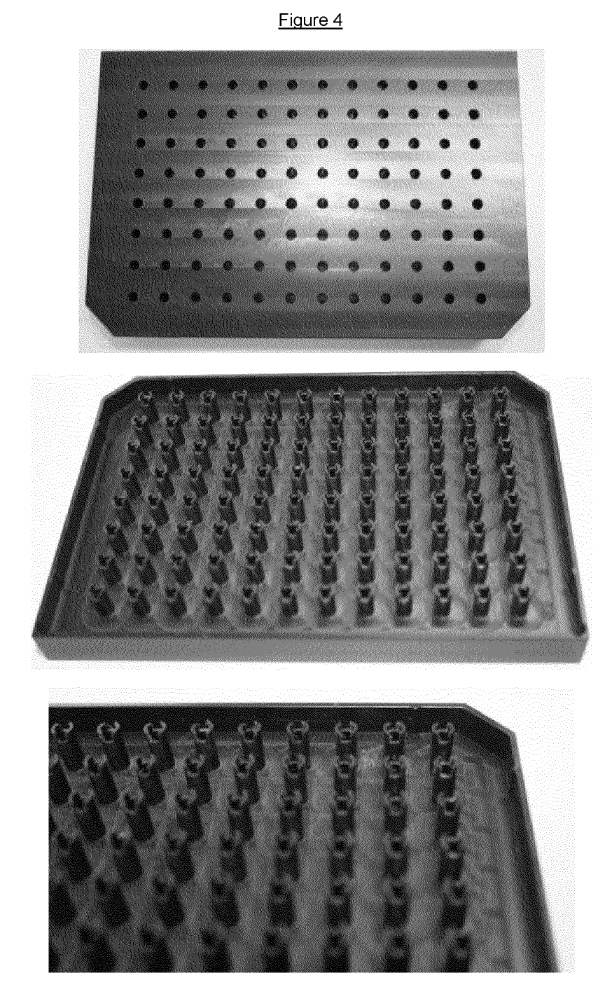

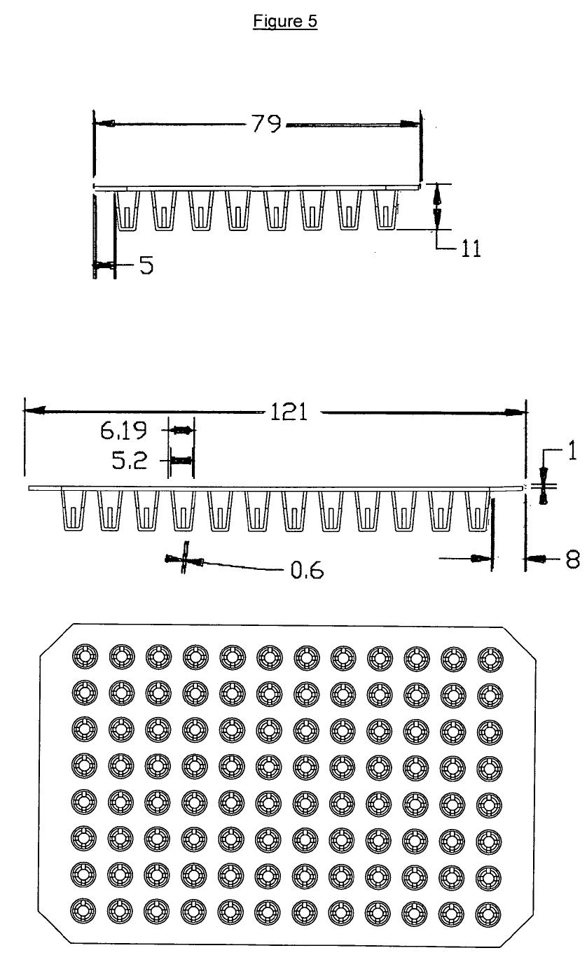

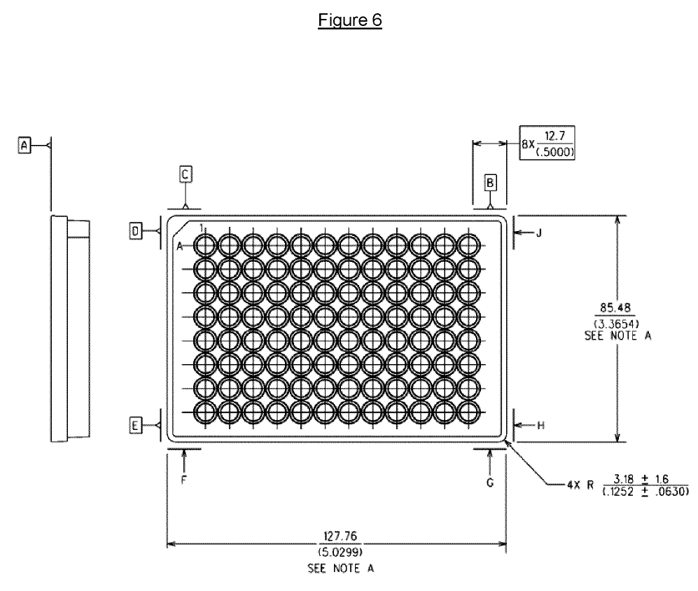

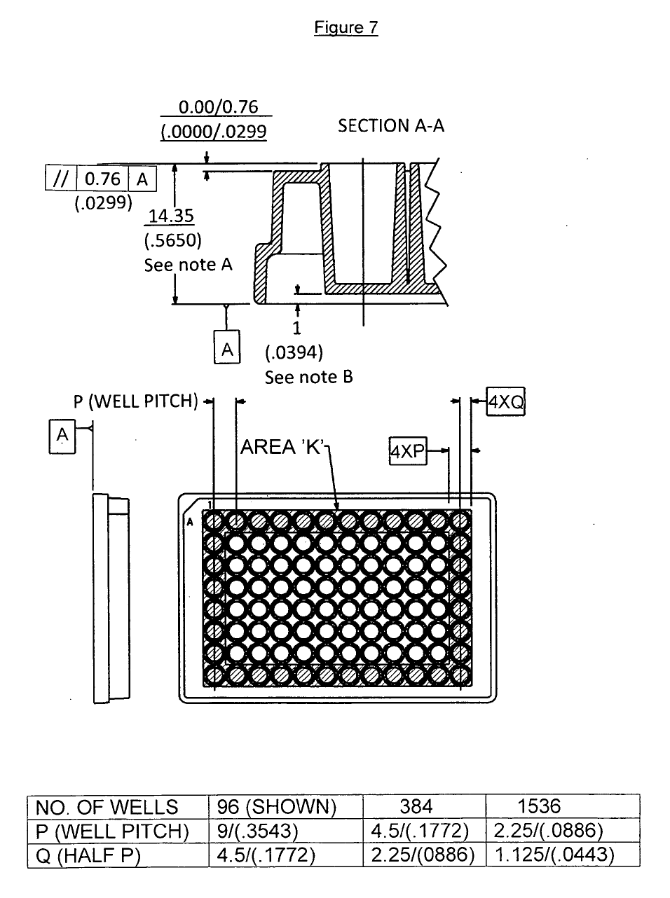

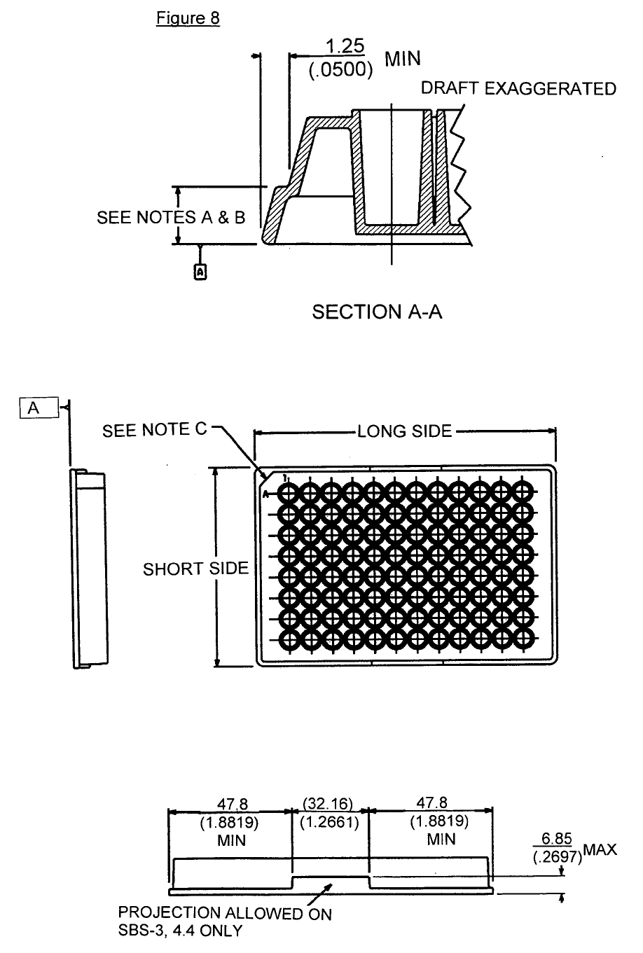

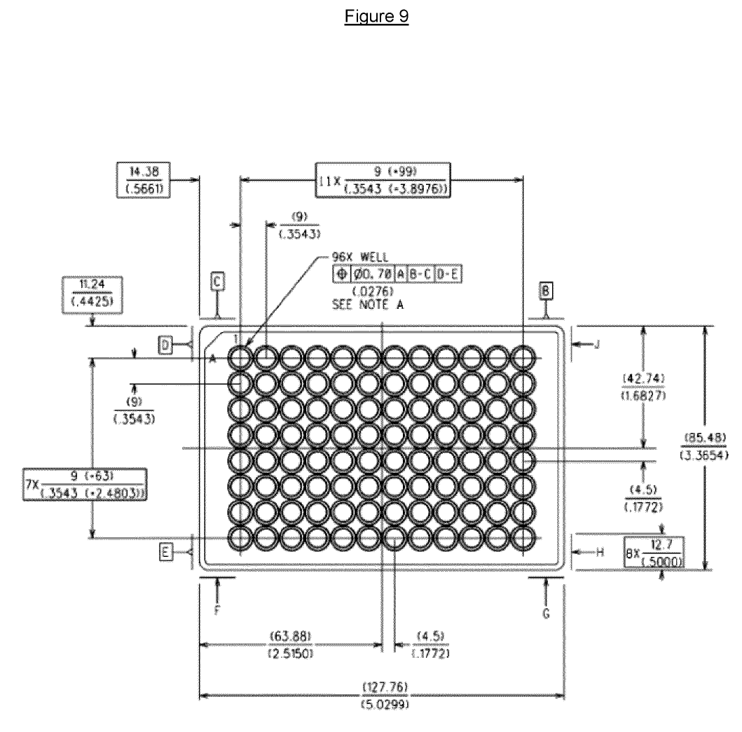

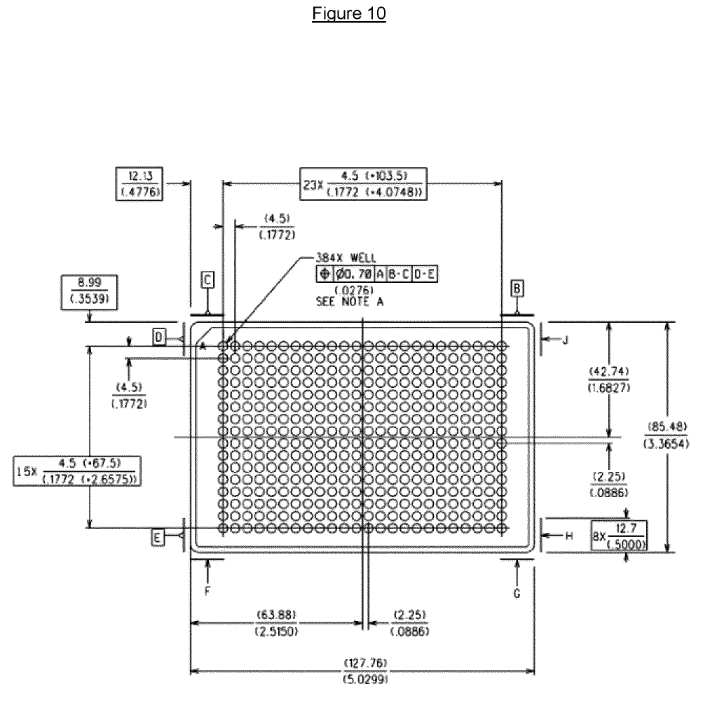

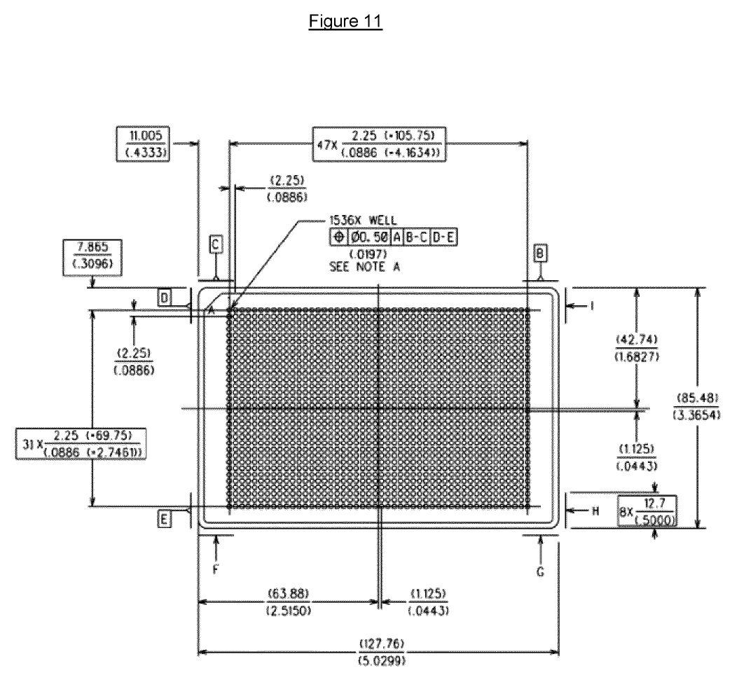

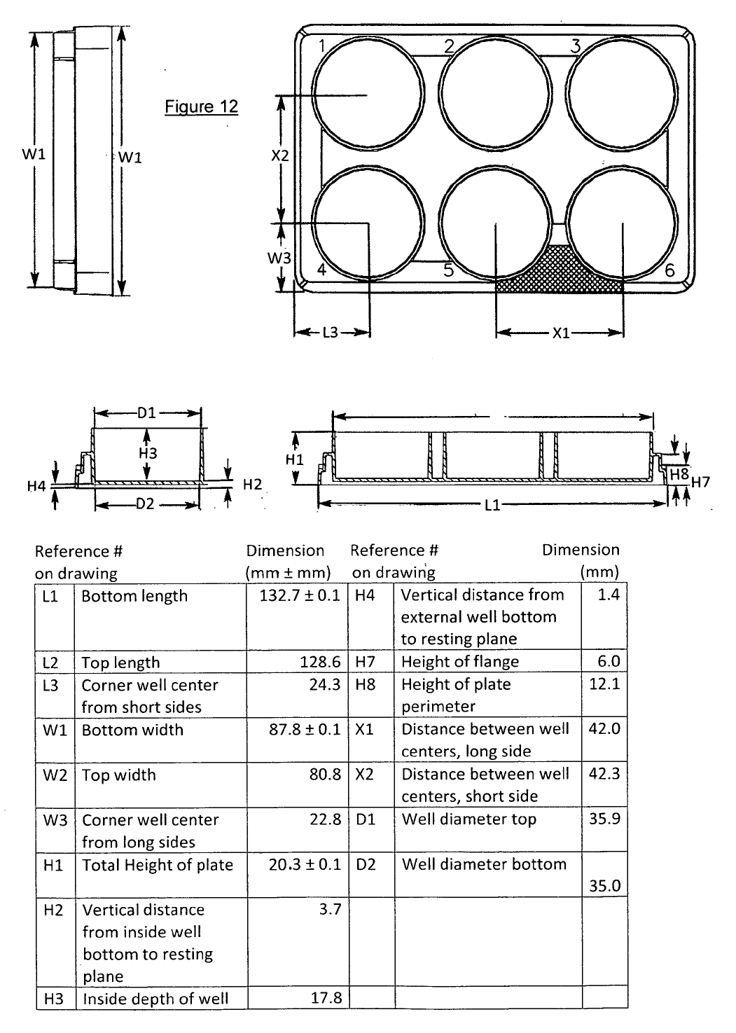

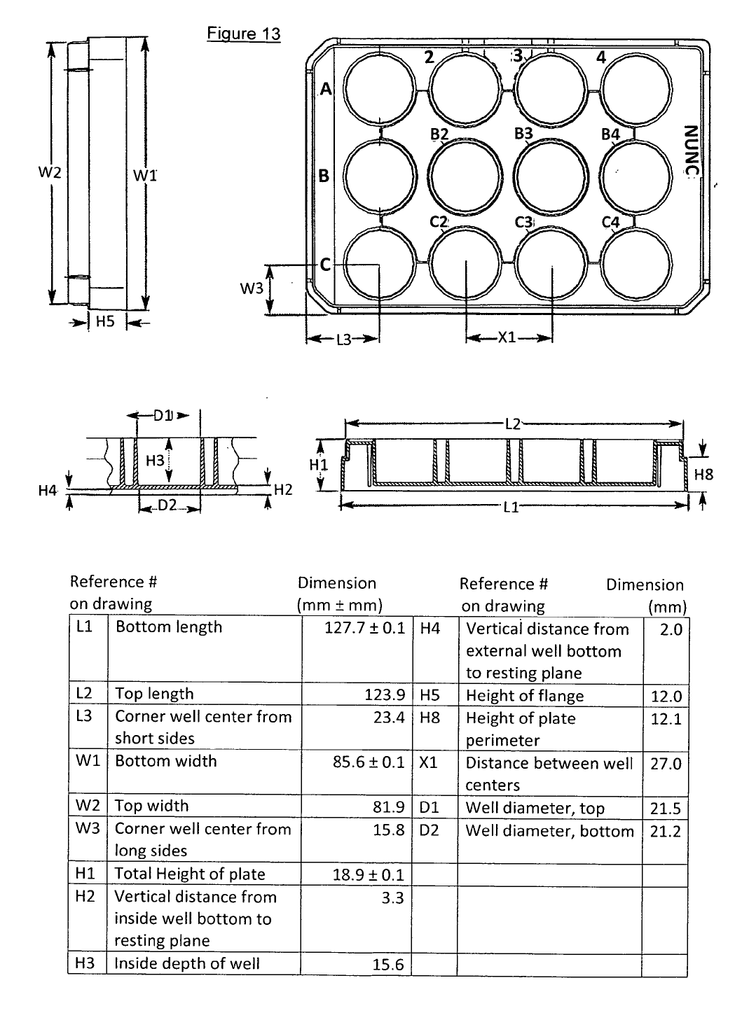

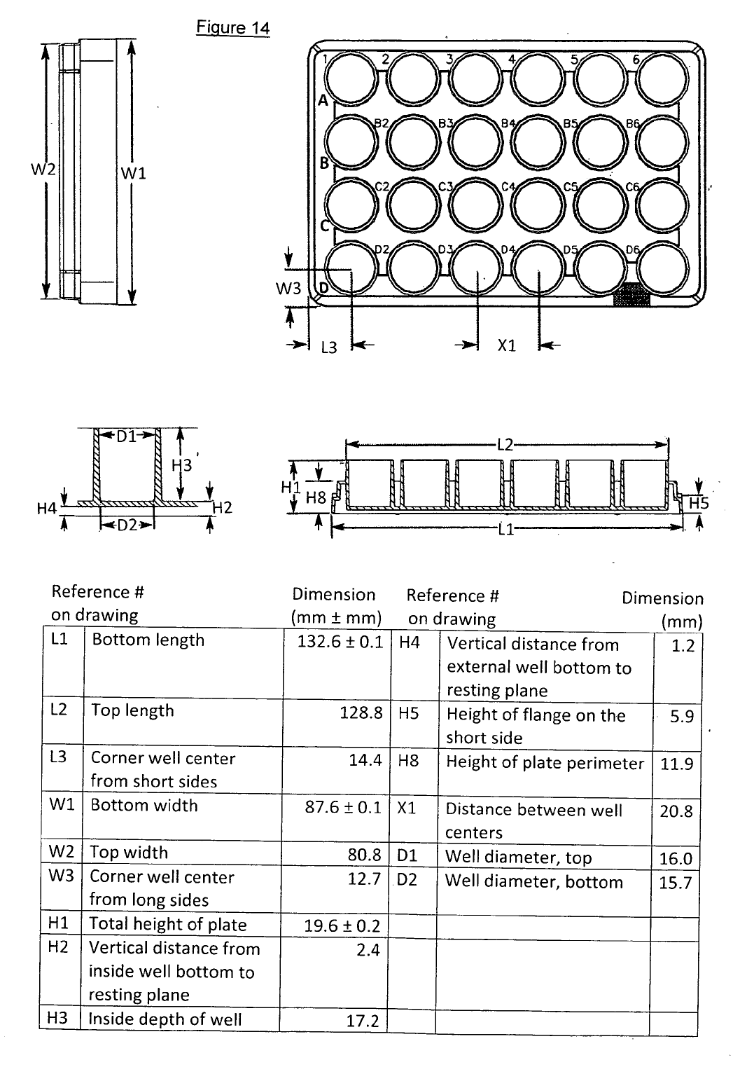

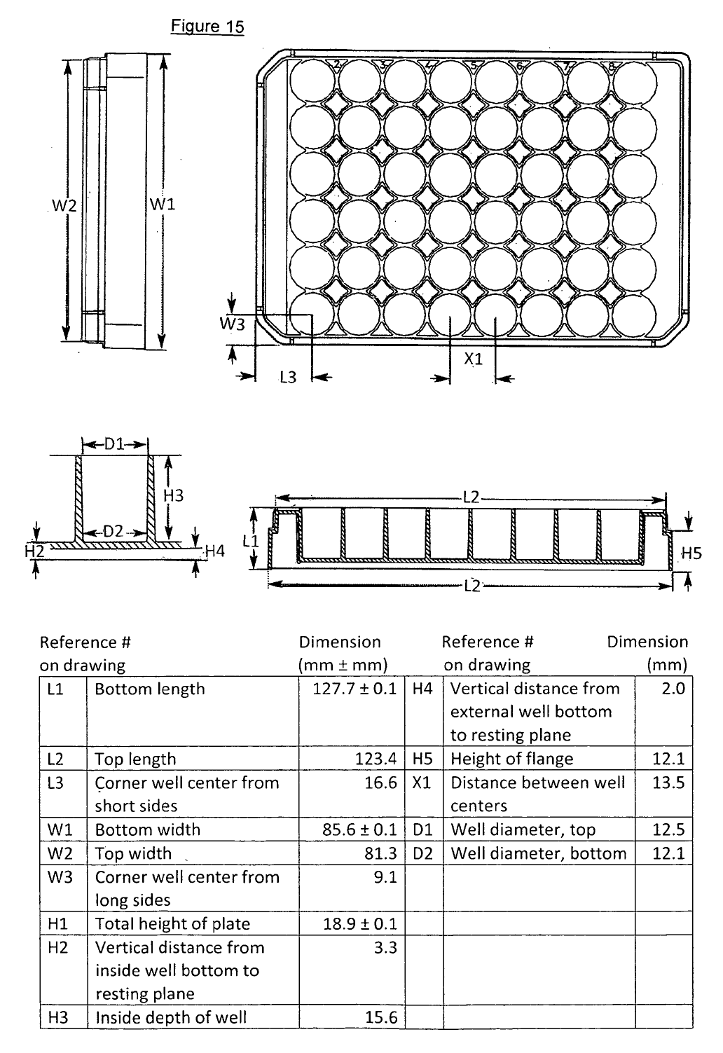

The prototype adapter shown in FIG. 4 was placed on top of the microtiter plate and pressed down as far as possible. The adapter fingers pressed the biomass mats down into the liquid in each well, and the liquid flowed around the mats and into the hollow adapter fingers, where it was available for sampling with no risk of biomass blocking or clogging the tip of sampling pipette or needle. No liquid was forced out of the wells by the adapter and no transfer of liquid from one well to another was observed. 100 microliter cell-free supernatant was then successfully withdrawn by pipette from each hollow adapter finger for further analysis.

This result is particularly useful for automated high throughput screening setups, where a high degree of reliability and reproducibility is very important.

The microtiter plate fitted with the adapter may be easily refrigerated or frozen as one for future testing.

### Example 2

### Adapter-Enabled Biomass Transfer from Microtiter Plate with Aspergillus oryzae

The adapter described in Example 1 was tested with A. oryzae grown in 96-well microtiter plates. The adapter could not only be used as a biomass presser to push biomass to the bottom of microplate wells to allow sampling of the liquid supernatant, as shown above, it could also be used for transferring biomass into a new microplate, e.g., for storage, while leaving the spent growth medium or supernatant in the original microplate accessible for easy sampling and further assays.

A prototype of an adapter according to the invention was manufactured by 3D printing (stereolithography) after the drawings in FIG. 5; photos of the resulting unit are shown in FIG. 18.

Our first prototype shown in FIG. 4 was designed for use in a robot with fixed (thin) tips—we would like to be able to use it with disposable tips. Disposable tips are often larger and have a conical shape—so the shape and diameter of the protruding fingers in the second prototype should be a bit different/larger, at least in the top to fit the conical shape of the disposable pipette tips, as shown in FIGS. 5 and 18.

In the second prototype we also removed the fixed side-panels or flanges of the unit in order to use a standard microtiter plate lid on top of the microplate with the fitted adapter. In this way, the microtiter plate w. adapter can be used in our standard screening setup—it is possible to cover the microplate with a standard lid before/after analysis and during incubation for e.g. growth.

For all experiments A. oryzae was grown for 4 days at 37° C. in 96-well MTP, containing 200 μl of broths (three different kinds were tested: YP broth, 2XSC+2% maltose, Sucrose/NaNO₃ agar without agar.

First, the adapter was applied on top of a microplate after growth of A. oryzae, which had formed a surface layer of biomass, and the adapter was then carefully pressed down. The biomass stayed as a compact layer on the bottom of each well after having been pressed down, while clear supernatant could be withdrawn from the hollow of the finger within each well of the microplate. Approximately 2×50 μl was withdrawn successfully from each well by our Hamilton 4200 robot system. For recovery of strain isolates, new broth was added from above to each finger while still being placed in the microplate.

Secondly, the adapter was placed onto a microplate just after its inoculation and the system was then incubated for growth as described above. After growth, all the surface layer biomass was retained in the hollow of the fingers in the microplate wells. The adapter was carefully removed and transferred onto a new microplate—either empty for storage or filled with fresh broth for re-growth.

Analysis of enzyme (lipase) expressed in A. oryzae, when employing the two different ways of using the adapter, did not show any significant difference in the concentrations of lipase in the supernatants as determined by ELISA (not shown). Note: Expression was only analysed in 2×SC+2% maltose.

There was a tendency of lipase expression being a little bit lower in the microtiter plate where the adapter was present during growth (when it was used as a bio-transfer unit). However, the biological variation (stdev) was also lower (13% vs. 26% upon usage as biomass-transporter and biomass-presser, respectively) and the expression was still very good for assaying.

We are in the process of developing the adapter of the invention further. Addition of an upwards skirt or flange (approximately 4-5 mm) to the upper part will make the adapter easier to handle both for human hands and robots. This will also raise a microplate lid, so the lid will not come in contact with the adapter surface, thereby eliminating the possibility of condensation on the inside of the lid, which might crosscontaminate the wells.

1. A microplate adapter for sampling a liquid growth medium from a microplate, where one or more microorganism has formed a layer of biomass on the surface of the liquid growth medium in one or more well, said adapter comprising a main body [FIG. 1: 1] with a plurality of hollow protruding fingers [FIG. 1: 3], wherein:
(a) each finger protrudes into a corresponding well of the microplate when the adapter is placed on top of the microplate, whereby said finger pushes any biomass layer on the surface down into the liquid growth medium;
(b) each finger has one or more openings in its bottom and/or sides to allow free flow of liquid from the well into the hollow of each finger, when the adapter is placed on top of the microplate and the surface layer of biomass is pushed down; and
(c) each finger has one or more openings at the top as well as internal dimensions that allow individual samples to be taken of the liquid in the hollow of each finger, when the adapter is placed on top of the microplate.

2. The microplate adapter of claim 1, which is suitable for a 6, 12, 24, 48, 96, 384 or 1536 well microplate; preferably the microplate meets the Standards ANSI/SBS 1-2004 through ANSI/SBS 4-2004.

3. The microplate adapter of claim 1 the protruding fingers of which [FIG. 1: 3] extend anywhere from at least 1 mm into the corresponding wells, when the adapter is placed on top of the microplate; preferably the fingers extend at least 2, 3, 4, 5, 6, 7, 8, 9, 10, 11, 12, 13 or even 14 mm into the corresponding wells, when the adapter is placed on top of the microplate

4. The microplate adapter of claim 1, wherein the protruding fingers [FIG. 1: 3] are tapered or conical on the outside, thereby allowing easier insertion of the fingers into the wells of the microplate; preferably the sides are angled between 0.1 and 20 degrees.

5. The microplate adapter of any of claim 1, wherein the protruding fingers [FIG. 1: 3] are tapered or conical on the inside, thereby allowing easier sampling or insertion of a disposable pipette tip for sampling; preferably the sides are angled between 0.1 and 20 degrees.

6. The microplate adapter of any of claim 1, which has one or more sides that fit around the outside of the microplate; preferably the sides are at least 1 mm high, more preferably at least 2, 3, 4, 5, 6, 7, 8, 9, 10 mm high.

7. The microplate adapter of claim 1, which has one or more chamfer corresponding to any chamfer on the microplate.

8. A method of sampling a liquid growth medium from a microplate, where one or more microorganism has formed a layer of biomass on the surface of the liquid growth medium in one or more well, said method comprising the steps of:
A) placing an adapter on top of the microplate, said adapter comprising a main body [FIG. 1: 1] with a plurality of hollow protruding fingers [FIG. 1: 3], wherein:
(i) each finger protrudes into a corresponding well of the microplate when the adapter is placed on top of the microplate, whereby said finger pushes any biomass layer on the surface down into the liquid growth medium;
(ii) each finger has one or more openings in its bottom and/or sides to allow free flow of liquid from the well into the hollow of each finger, when the adapter is placed on top of the microplate and the surface layer of biomass is pushed down; and
(iii) each finger has one or more openings at the top as well as internal dimensions that allow individual samples to be taken of the liquid in the hollow of each finger, when the adapter is placed on top of the microplate; and
B) sampling the liquid growth medium from the hollow of at least one finger.

9. The method of claim 8, wherein the microplate adapter is suitable for a 6, 12, 24, 48, 96, 384 or 1536 well microplate; preferably the microplate meets the Standards ANSI/SBS 1-2004 through ANSI/SBS 4-2004.

10. The method of claim 8, wherein the protruding fingers [FIG. 1: 3] extend anywhere from at least 1 mm into the corresponding wells, when the adapter is placed on top of the microplate; preferably the fingers extend at least 2, 3, 4, 5, 6, 7, 8, 9, 10, 11, 12, 13 or even 14 mm into the corresponding wells, when the adapter is placed on top of the microplate

11. The method of any of claim 8, wherein the protruding fingers [FIG. 1: 3] are tapered or conical on the outside, thereby allowing easier insertion of the fingers into the wells of the microplate; preferably the sides are angled between 0.1 and 20 degrees.

12. The method of any of claim 8, wherein the protruding fingers [FIG. 1: 3] are tapered or conical on the inside, thereby allowing easier sampling or insertion of a disposable pipette tip for sampling; preferably the sides are angled between 0.1 and 20 degrees.

13. The method of any of claim 8, wherein the microplate adapter has one or more sides that fit around the outside of the microplate; preferably the sides are at least 1 mm high, more preferably at least 2, 3, 4, 5, 6, 7, 8, 9, 10 mm high.

14. A method for transferring a surface-growing microorganism from the surface of liquid medium in all wells of a microplate simultaneously, said method comprising:
A) placing an adapter on top of the microplate, said adapter comprising a main body [FIG. 1: 1] with a plurality of hollow protruding fingers [FIG. 1: 3], so that:
(i) each finger protrudes into a corresponding well of the microplate;
(ii) each finger has one or more openings in its bottom and/or sides to allow free flow of liquid from the well into and out of the hollow of each finger, when the adapter is placed on top of the microplate or removed from it; and
(iii) each finger has one or more openings at the top to allow air to escape;
wherein a liquid growth medium and a microorganism has been added to the wells of the microplate before, during or after step (A), but before step (B);
B) incubating the microplate and adapter together under conditions suitable for cultivating the microorganism, which then forms a layer of biomass on the surface of the liquid growth medium in the hollow of the protruding fingers;
C) removing the adapter from the microplate, so the liquid medium drains from the hollow of each finger while the surface-layer of biomass remains inside each finger, whereby the microorganisms from all the wells become transferable at once inside the fingers of the adapter, to be further processed or stored.

15. The method of claim 14, comprising an additional step D) sampling the spent liquid growth medium in one or more wells of the microplate.

16. The method of claim 14, wherein the microplate adapter is suitable for a 6, 12, 24, 48, 96, 384 or 1536 well microplate; preferably the microplate meets the Standards ANSI/SBS 1-2004 through ANSI/SBS 4-2004.

17. The method of any of claim 14, wherein the protruding fingers [FIG. 1: 3] extend anywhere from at least 1 mm into the corresponding wells, when the adapter is placed on top of the microplate; preferably the fingers extend at least 2, 3, 4, 5, 6, 7, 8, 9, 10, 11, 12, 13 or even 14 mm into the corresponding wells, when the adapter is placed on top of the microplate

18. The method of any of claim 14, wherein the protruding fingers [FIG. 1: 3] are tapered or conical on the outside, thereby allowing easier insertion of the fingers into the wells of the microplate; preferably the sides are angled between 0.1 and 20 degrees.

19. The method of any of claim 14, wherein the protruding fingers [FIG. 1: 3] are tapered or conical on the inside, thereby allowing easier sampling or insertion of a disposable pipette tip for sampling; preferably the sides are angled between 0.1 and 20 degrees.

20. The method of any of claim 14, wherein the microplate adapter has one or more sides that fit around the outside of the microplate;
preferably the sides are at least 1 mm high, more preferably at least 2, 3, 4, 5, 6, 7, 8, 9, 10 mm high.
